(19) United States
(12) Patent Application Publication
(10) Pub. No.: US 2007/0071756 A1
(43) Pub. Date: Mar. 29, 2007

(54) Delivery of an agent to ameliorate inflammation

(76) Inventor: Gholam A. Peyman, Sun City, AZ (US)

Correspondence Address: WOOD, HERRON & EVANS, LLP, 2700 CAREW TOWER, 441 VINE STREET, CINCINNATI, OH, 45202, US

(21) Appl. No.: 11/348,017

(22) Filed: Feb. 6, 2006

Related U.S. Application Data
(63) Continuation-in-part of application No. 11/234,970, filed on Sep. 26, 2005.

(51) Int. Cl. A61K 48/00; A61K 39/395; A61K 31/56

(52) U.S. Cl. 424/155.1; 424/145.1; 514/44; 514/171

(57) ABSTRACT

A method delivering an anti-vascular endothelial growth factor (VEGF) agent to ameliorate inflammation at a site in the body that may be the eye, a joint, the brain, etc. or to reduce corneal neovascularization. In one embodiment, one or more other agents, such as non-steroidal anti-inflammatory agents, steroids, etc., may be included with the anti-VEGF agent. The anti-VEGF agent may be bevacizumab, ranibizumab, sunitinib maleate, pegaptanib, etc.

This application is a Continuation-in-Part of U.S. application Ser. No. 11/234,970, filed on Sep. 26, 2005 which is expressly incorporated by reference herein in its entirety.

This application is related to commonly assigned, copending applications, Serial Numbers unknown, each filed Feb. 6, 2006 and entitled DEVICE FOR DELIVERY OF AN AGENT TO THE EYE AND OTHER SITES, and DELIVERY OF AN OCULAR AGENT, each naming Peyman as the inventor, each of which is expressly incorporated by reference herein in its entirety.

This application contains at least one drawing executed in color. A Petition under 37 C.F.R. §1.84 requesting acceptance of the color drawings is filed separately on even date herewith. Copies of this patent with color drawing(s) will be provided by the Office upon request and payment of the necessary fee.

A method is disclosed for controlling, reducing, or preventing inflammation, an anti-inflammatory response, and/or effects of an anti-inflammatory response, encompassed generally as ameliorating inflammation. The method provides to a patient an anti-vascular endothelial growth factor (VEGF) agent to ameliorate inflammation. Anti-VEGF agents include but are not limited to bevacizumab (rhuMab VEGF, Avastin®, Genentech, South San Francisco, Calif.), ranibizumab (rhuFAb V2, Lucentis®, Genentech), pegaptanib (Macugen®, Eyetech Pharmaceuticals, New York N.Y.), sunitinib maleate (Sutent®, Pfizer, Groton, Conn.), TNP470, integrin av antagonists, 2-methoxyestradiol, paclitaxel, and P38 mitogen activated protein kinase inhibitors. Anti-VEGF RNA (short double-stranded RNA to trigger RNA interference and thereby impair VEGF synthesis) may also be used as an anti-VEGF agent.

In one embodiment, the anti-VEGF agent is bevacizumab, administered either alone or with one or more agent(s) known to one skilled in the art under the classification of anti-inflammatory agents. These include, but are not limited to, steroids, anti-prostaglandins, matrix metalloproteinase inhibitors, non-steroidal anti-inflammatory drugs (NSAIDS), macrolides, anti-proliferative agents, anti-cancer agents, etc. In one embodiment, the method ameliorates inflammation using the anti-VEGF agent such as bevacizumab alone. In another embodiment, the method ameliorates inflammation using the anti-VEGF agent such as bevacizumab to supplement known anti-inflammatory agents. In both embodiments, the method ameliorates inflammation at any stage, even early stage inflammation before occurrence of an angiogenic component. The method controls inflammation, and counteracts the action of angiogenic agents such as VEGF on the permeability of a vessel wall, thereby reducing or preventing the resulting tissue damage due to fluid leakage from the vessel (extravasation). The method is applicable to any tissue or site in the body, and to any cause of inflammation such as immune disease including auto immune disease, viral and/or bacterial infection, trauma including surgical trauma, etc. In one embodiment, the method controls, reduces, or prevents tissue damage in the brain. In one embodiment, the method controls, reduces, or prevents tissue damage in the eye.

Inflammation is a localized, protective response of vascularized tissue to sub-lethal tissue injury or destruction. The response functions to destroy, dilute, or sequester both the injurious agent and the injured tissue. Inflammation can be classified according to duration as either acute or chronic. In the acute form of an inflammatory response, classical signs are pain, heat, redness, swelling, and loss of function. Histologically, there are a complex series of events including dilatation of arterioles, capillaries and venules, with increased permeability and blood flow, exudation of fluids including plasma proteins, and leukocyte migration and accumulation at the site of injury. This reaction may trigger a systemic response such as fever, leukocytosis, protein catabolism, and altered hepatic synthesis of plasma proteins such as C-reactive protein. Chronic inflammation is characterized by macrophage and lymphocyte infiltration into the affected and surrounding tissue.

Inflammation is a homeostatic response to tissue damage by a range of stimuli, including infection and trauma. For example, an inflammatory response helps to destroy or inactivate invading pathogens. In cases of auto immune diseases such as rheumatoid arthritis, etc., inflammation is a response against self. The inflammatory process removes waste and debris and restores normal function, either through resolution or repair. Tissue structure is normal after resolution, whereas repair leads to a functional, but morphologically altered, organ. In acute inflammation, tissue damage is followed by resolution or healing by scar formation, whereas in chronic inflammation, damage and repair continue concurrently. The initial inflammatory response is usually acute, and may or may not evolve into chronic inflammation. However, chronic inflammation is not always preceded by an acute phase. Although usually beneficial to the organism, inflammation itself may lead to tissue damage, resulting in escalation of chronic inflammation. Inflammation underlies the pathology of virtually all rheumatologic diseases. The severity of disorders, such as arthritis, is classified according to the degree of inflammation and its destructive effects.

Anti-VEGF agents affect the process of angiogenesis, which is the growth of new blood vessels from pre-existing vasculature. It is a fundamental process required for embryogenesis, growth, tissue repair after injury, and the female reproductive cycle. It also contributes to the pathology of conditions such as cancer, age related macular degeneration, psoriasis, diabetic retinopathy, and chronic inflammatory diseases in joints or lungs. Angiogenesis is stimulated when hypoxic, diseased, or injured tissues produce and release angiogenic promoters such as VEGF, platelet derived growth factor (PDGF), or fibroblast growth factor (FGF)-1. These angiogenic factors stimulate the migration and proliferation of endothelial cells in existing vessels and, subsequently, the formation of capillary tubes and the recruitment of other cell types to generate and stabilize new blood vessels.

Angiogenic factors may be pro-inflammatory factors. Relatively minor irritation of internal tissues, such as occurs during surgery, does not lead to neovascularization, but encourages tissue adhesion and scarring. Agents that inhibit angiogenesis such as the previously disclosed TNP470, integrin av antagonists, 2-methoxyestradiol, paclitaxel, P38 mitogen activated protein kinase inhibitors, anti-VEGF siRNA, and sunitinib maleate (Sutent®/SU11248) may inhibit synovitis, uveitis, iritis, retinal vasculitis, optic nerve neuritis, papillitis, retinitis proliferance in diabetes, etc. Expression of adhesion molecules such as integrin avb3 and e-selectin are upregulated in new vessels, and new vessels appear sensitive to inflammogens. The angiogenic factor FGF-1 enhances antigen-induced synovitis in rabbits, but is not pro-inflammatory when administered alone. However, angiogenesis occurs in the absence of inflammation such as during embryonic growth and in the female reproductive cycle. Thus, inflammation and angiogenesis can occur independently and administration of anti-VEGF agents such as bevacizumab, either alone or to supplement known anti-inflammatory agents, ameliorates both inflammation without an angiogenic component (earlier stage inflammation), and inflammation that has progressed to an angiogenic component (later stage inflammation). Coexistence of inflammation and angiogenesis may lead to more severe, damaging, and persistent inflammation.

Angiogenesis enhances tumor growth, and anti-angiogenic agents are used clinically. Mechanisms by which new vessels enhance tumor growth include providing metabolic requirements of the tumor, generating growth factors by vascular cells, and inhibiting apoptosis. Inhibiting the function of growth factors such as VEGF can reduce or prevent pathological angiogenesis in tumors.

Angiogenesis may also contribute to thickening of airways in asthma and of lung parenchyma in pulmonary fibrosis, and to growth of sarcoid granulomas. Growth of granulation tissue into airspaces also may be angiogenesis-dependent in bronchi after lung transplant and in alveoli after acute lung injury or in other forms of pulmonary fibrosis. Angiogenesis may also contribute to growth of the synovial pannus in rheumatoid arthritis. Interposition of expanded, innervated synovium between articulating surfaces may contribute to pain on movement. In each of these situations, the expanded tissue may impair function.

The new blood vessels that result from angiogenesis have incomplete walls and are particularly susceptible to disruption and fluid extravasation. This has been proposed as a cause of pulmonary hemorrhage in inflammatory lung disease. Hemosiderin deposits and extravasated erythrocytes are commonly present in inflammatory synovitis, although the contribution of angiogenesis to synovial microhemorrhage is unknown, and its contribution to synovial inflammation remains unclear. The inflammatory potential is evident, however, in patients with hemophilia.

Angiogenesis occurs as an orderly series of events, beginning with production and release of angiogenic growth factors (proteins) that diffuse into nearby tissues. The angiogenic growth factors bind to specific receptors located on the endothelial cells of nearby preexisting blood vessels. Once growth factors bind to their receptors, the endothelial cells are activated and begin to produce enzymes and other molecules that dissolve tiny holes in the sheath-like basement membrane that surrounds existing blood vessels. The endothelial cells begin to divide and proliferate, and they migrate through the holes of the existing vessel towards the diseased tissue or tumor. Specialized adhesion molecules or integrins (avb3, avb5) help to pull the new blood vessels forward. Additional enzymes, termed matrix metalloproteinases (MMP), are produced and dissolve the tissue in front of the sprouting vessel tip in order to accommodate it. As the vessel extends, the tissue is remolded around the vessel. Sprouting endothelial cells roll up to form a blood vessel tube and individual blood vessel tubes connect to form blood vessel loops that can circulate blood. The newly formed blood vessel tubes are stabilized by smooth muscle cells, pericytes, fibroblasts, and glial cells that provide structural support, permitting blood flow to begin.

VEGF is a specific angiogenesis growth factor that binds to receptors on blood vessels and stimulates the formation of new blood vessels. VEGF is a potent inducer of both endothelial cell proliferation and migration, and its biologic activities are largely specific for endothelial and vascular smooth muscle cells. Unlike basic fibroblast growth factor (bFGF), high levels of VEGF are not present in early surgical wounds. Rather, VEGF levels peak seven days after the wound is created, at which point VEGF appears to be a major stimulus for sustained induction of blood vessel growth and high levels of PDGF have been shown. There are abundant sources of VEGF in wounds. Many cell types produce VEGF, including keratinocytes, macrophages, fibroblasts, and endothelial cells. Thus, there is massive VEGF secretion, particularly in the setting of hypoxia, which is often observed in wounds.

Anti-VEGF agents inhibit the action of VEGF. As one example of an anti-VEGF agent, bevacizumab is a recombinant humanized monoclonal IgG1 antibody that binds to and inhibits the biologic activity of human VEGF in in vitro and in vivo assay systems by preventing binding of VEGF with its receptor on the surface of vascular endothelial cells, thus preventing endothelial cell proliferation and new vessel formation. Bevacizumab contains human framework regions and the complementarity-determining regions of a murine antibody that binds to VEGF; it has a molecular weight of about 149 kilodaltons. Bevacizumab, by binding to VEGF, blocks VEGF from binding to receptors and thus blocks angiogenesis. Bevacizumab is typically administered by intravenous infusion, diluted in 0.9% sodium chloride for injection from a 25 mg/ml preparation.

Ranibizumab is a derivative of the full-length antibody bevacizumab (Fab fragment), and is further modified to increase its affinity for VEGF. Both bevacizumab and ranibizumab bind all biologically active isoforms and proteolytic fragments of VEGF, but there are differences. Monovalent binding of a Fab fragment such as ranibizumab to its target antigen would not force the target to dimerize, and hence is useful to manipulate cell receptor function, but its effective antigen binding capacity is lower than its full antibody counterpart. However, VEGF, which is the desired target, is a soluble factor and not a cellular receptor. Therefore, the increased effective binding by the full length antibody bevacizumab enhances inhibition of the VEGF signal and thus provides an enhanced anti-angiogenic effect. Bevacizumab has also been “humanized” to decrease any antigenic effect it may have on the patient, and bevacizumab has a higher molecular weight; this full-length antibody likely will not penetrate the retina to the same extent as the lower molecular weight fragment ranibizumab. However, the increased size of bevacizumab may decrease its clearance rate from the site of action.

Among the available anti-inflammatory agents, many have a target of action to block or ameliorate the actions of pro-inflammatory signals, such as histamine and cytokines. Although this provides some relief from the harmful effects of inflammation, it does not address the cause of the problem. Leukocytes and macrophages, which release pro-inflammatory factors into affected areas, are allowed access to the inflamed tissue following new blood vessel formation.

In one embodiment, the inventive method administers one or a combination of anti-VEGF agent(s) such as bevacizumab, ranibizumab, pegaptanib, etc. as the sole agent(s) to ameliorate inflammation, and thus to control, reduce or prevent an inflammatory response or ameliorate the effects of an inflammatory response. In one embodiment, bevacizumab is used to enhance reabsorption of inflammatory exudates. Decreasing the level of exudates in the eye reduces the inflammatory process and the ensuing hyperpermeable state that occurs with allergies, infection, responses to ocular photodynamic therapy (PDT) and laser treatments, after ocular surgery or trauma, etc. In one embodiment, the anti-VEGF agent is administered to ameliorate an inflammatory process without an angiogenic component. Many inflammatory processes, such as early stage inflammation, are not associated with the formation of new blood vessels. Examples include, but are not limited to, inflammatory diseases of the central nervous system (brain and spinal cord) such as abscess, meningitis, encephalitis, vasculitis, and conditions resulting in cerebral edema; inflammatory diseases of the eye (uveitis, subsequently discussed), macular edema, and others known to one skilled in the art.

In one embodiment, the anti-VEGF agent is administered to ameliorate the scarring and adhesions that are a part of the inflammatory process. Adhesions are bands of scar tissue that bind two internal body surfaces. They are an inflammatory response to tissue damage, and occur as a normal part of any healing process. As one example, adhesions frequently occur during the post-surgical healing process during which tissues have experienced mechanical trauma. However, adverse effects can occur when internal surfaces bind, and adhesions may persist even after the original trauma has healed. Surgery to repair adhesions itself results in recurrent or additional adhesions. The presence of adhesions may also complicate surgical procedures, for example, ocular conjunctival adhesions may complicate subsequent glaucoma surgery.

Adhesions can occur following any type of trauma or surgery, including but not limited to ocular surgery. Examples of ocular surgery that may result in adhesions include glaucoma filtration operations (i.e., iridencleisis and trephination, pressure control valves), extraocular muscle surgery, diathermy or scleral buckling surgery for retinal detachment, and vitreous surgery. Examples of ocular trauma include penetrating ocular injuries, intraocular foreign body, procedures such as PDT, scatter laser threshold coagulation, refractive surgery, and blunt trauma.

In one embodiment, anti-VEGF agents ameliorate disorders with both a vascular proliferative component and a scarring component. As one example, the invention may be used in patients with the ocular disease pterygia. In these patients, fibrovascular proliferation results in scarring of the conjunctiva. An elevated, superficial, external ocular mass, termed a pterygium, forms and extends onto the corneal surface. Patients may experience symptoms of inflammation (e.g., redness, swelling, itching, irritation) and blurred vision. The mass itself may become inflamed, resulting in redness and ocular irritation. Left untreated, pterygia can distort the corneal topography, obscure the optical center of the comea, and result in altered vision.

The process whereby scar tissue forms (scarring) can occur without new blood vessels being formed (neovascularization). However, the neovascularization process always results in scarring because of the cell proliferation that occurs with the formation of new vessels also results in the proliferation of fibroblasts, glial cells, etc. that result in scar tissue formation. The inventive method may be used to ameliorate the scarring process.

In one embodiment, the anti-VEGF agent is administered to ameliorate inflammation of uveal tissues (uveitis, an inflammation of tissues in the middle layer of the eye, mainly the iris (iritis) and the ciliary body). Ocular inflammation may be associated with underlying systemic disease or autoimmunity, or may occur as a direct result of ocular trauma or infectious agents (bacterial, viral, fungal, etc.). Inflammatory reactions in adjacent tissues, e.g., keratitis, can induce a secondary uveitis. There are both acute and chronic forms of uveitis. The chronic form is frequently associated with many systemic disorders and most likely occurs due to immunopathological mechanisms.

Uveitis presents with ocular pain, photophobia and hyperlacrimation, with decreased visual acuity ranging from mild blur to significant vision loss. Hallmark signs of anterior uveitis are cells and flare in the anterior chamber. If the anterior chamber reaction is significant, small gray to brown endothelial deposits known as keratic precipitates may arise, leading to endothelial cell dysfunction and corneal edema. There may be adhesions to the lens capsule (posterior synechia) or the peripheral cornea (anterior synechia). Granulomatous nodules may appear on the surface of the iris stroma. Intraocular pressure is initially reduced due to secretory hypotony of the ciliary body but, as the reaction persists, inflammatory by-products may accumulate in the trabeculum. If this debris builds significantly, and if the ciliary body resumes its normal secretory output, the pressure may rise sharply, resulting in a secondary uveitic glaucoma.

One skilled in the art will appreciate that scarring and adhesions in areas of the body other than the eye may be treated with the inventive method. Examples include adhesions associated with cardiac surgery (e.g., adhesions in the pericardial space), pulmonary surgery (e.g., in the periplural space), abdominal surgery (e.g., appendectomy, gastric bypass surgery), gynecological surgery (e.g., episiotomy, Caesarean section, hysterectomy), any type of laparoscopy or laparotomy surgery, reconstructive surgery (cosmetic or therapeutic), organ removal (partial or complete), etc.

In another embodiment, the inventive method administers an anti-inflammatory agent simultaneously or concomitantly with an anti-VEGF agent such as bevacizumab and thus controls, reduces, or prevents an inflammatory response. Other anti-VEGF agents such as Lucentis®, Macugen®, Sutent®, geldanamycin, etc. may be included.

The method may be used for any tissue including, but not limited to, eye (e.g., to ameliorate conjunctivitis (inflammation of the conjunctivae, the mucous membranes covering the sclera and inner eyelid), that may be associated with bacterial, viral, or Chlamydia infections, allergies, or susceptibility to irritants such as chemicals, smoke, etc., lung (e.g., to ameliorate interstitial lung disease, inflammation of the interstitium (tissue between the air sacs in the lung)), bone (e.g., to ameliorate synovitis, inflammation of the synovium (the membranes lining joints) that may be associated with arthritis), brain (e.g., to ameliorate encephalitis (inflammation of brain tissue and/or membranes)), and muscle (e.g., to ameliorate myopathies (inflammation of muscles, such as muscles near a joint)). The method may be used on patients at risk for developing inflammation. The method may be used on patients with inflammation and/or inflammatory processes from any cause, including but not limited to auto immune diseases, diseases with an immune component, ischemic diseases, diabetes, age related macular degeneration, retinitis pigmentosa, infectious diseases, allergen-induced inflammation, other degenerative diseases, etc.

In the embodiment where the anti-VEGF agent(s) is administered with an anti-inflammatory agent, an effective amount of the anti-inflammatory agent is administered to a patient at a standard dose known to one skilled in the art. As one example, prednisone is administered for a systemic dose in the range between about 5 mg to about 100 mg daily. As another example, Solu-medrol® is administered intravenously in a single dose of about 1 mg. Other anti-inflammatory agents, possible routes of administration, doses, etc. are known to one skilled in the art. The agent may be administered by any route including enteral and parenteral route, for example, intravenously, orally, ocularly, etc. One skilled in the art will appreciate that the route of administration may vary due to factors such as agent solubility, patient needs, dose required, etc. The anti-inflammatory agent may be a fast-acting anti-inflammatory agent, a slow acting anti-inflammatory agent, or both a fast-acting and a slow-acting anti-inflammatory agent. The anti-inflammatory agent may be formulated for delayed and/or extended release to provide effects over a longer period of time.

Examples of anti-inflammatory agents recognized by one skilled in the art include, but are not limited to, the following: colchicine; a steroid such as triamcinolone (Aristocort®; Kenalog®), anecortave acetate (Alcon), betamethasone (Celestone®), budesonide cortisone, dexamethasone (Decadron-LA®; Decadron® phosphate; Maxidex® and Tobradex® (Alcon)), hydrocortisone methylprednisolone (Depo-Medrol®, Solu-Medrol®), prednisolone (prednisolone acetate, e.g., Pred Forte® (Allergan), Econopred and Econopred Plus® (Alcon), AK-Tate® (Akom), Pred Mild® (Allergan), prednisone sodium phosphate (Inflamase Mild and Inflamase Forte® (Ciba), Metreton® (Schering), AK-Pred® (Akorn)), fluorometholone (fluorometholone acetate (Flarex® (Alcon), Eflone®), fluorometholone alcohol (FML® and FML-Mild®, (Allergan), FluorOP®), rimexolone (Vexol® (Alcon)), medrysone alcohol (HMS® (Allergan)), lotoprednol etabonate (Lotemax® and Alrex® (Bausch & Lomb), and 11-desoxcortisol; an anti-prostaglandin such as indomethacin; ketorolac tromethamine; ((±)-5-benzoyl-2, 3-dihydro-1H-pyrrolizine-1-carboxylic acid, a compound with 2-amino-2-(hydroxymethyl)-1,3-propanediol (1:1) (Acular® Allegan), Ocufen® (flurbiprofen sodium 0.03%), meclofenamate, fluorbiprofen, and the pyrrolo-pyrrole group of non-steroidal anti-inflammatory drugs; a macrolide such as sirolimus (rapamycin), pimocrolous, tacrolimus (FK506), cyclosporine (Arrestase), everolimus 40-0-(2-hydroxymethylenrapamycin), ascomycin, erythromycin, azithromycin, clarithromycin, clindamycin, lincomycin, dirithromycin, josamycin, spiramycin, diacetyl-midecamycin, tylosin, roxithromycin, ABT-773, telithromycin, leucomycins, lincosamide, biolimus, ABT-578 (methylrapamycin), and derivatives of rapamycin such as temsirolimus (CCI-779, Wyeth) and AP23573 (Ariad); a non-steroidal anti-inflammatory drug such as derivatives of acetic acid (e.g. diclofenac and ketorolac (Toradol®, Voltaren®, Voltaren-XR®, Cataflam®)), salicylate (e.g., aspirin, Ecotrin®), proprionic acid (e.g., ibuprofen (Advil®, Motrin®, Medipren®, Nuprin®)), acetaminophen (Tylenol®), aniline (e.g., aminophenolacetaminophen, pyrazole (e.g., phenylbutazone), N-arylanthranilic acid (fenamates) (e.g., meclofenamate), indole (e.g., indomethacin (Indocin®, Indocin-SR®)), oxicam (e.g., piroxicam (Feldene®)), pyrrol-pyrrole group (e.g., Acular®), antiplatelet medications, choline magnesium salicylate (Trilisate®), cox-2 inhibitors (meloxicam (Mobic®)), diflunisal (Dolobid®), etodolac (Lodine®), fenoprofen (Nalfon®), flurbiprofen (Ansaid®), ketoprofen (Orudis®, Oruvail®), meclofenamate (Meclomen®), nabumetone (Relafen®), naproxen (Naprosyn®, Naprelan®, Anaprox®, Aleve®), oxaprozin (Daypro®), phenylbutazone (Butazolidine®), salsalate (Disalcid®, Salflex®), tolmetin (Tolectin®), valdecoxib (Bextra®), sulindac (Clinoril®), and flurbiprofin sodium (Ocufen®), an MMP inhibitor such as doxycycline, TIMP-1, TIMP-2, TIMP-3, TIMP-4; MMP1, MMP2, MMP3, Batimastat (BB-94), TAPI-2,10-phenanthroline, and marimastat. The composition may contain anti-PDGF compound(s) such as imatinib mesylate (Gleevec®), sunitinib malate (Sutent®) which has anti-PDGF activity in addition to anti-VEGF activity, and/or anti-leukotriene(s) such as genleuton, montelukast, cinalukast, zafirlukast, pranlukast, zileuton, BAYX1005, LY171883, and MK-571 to account for the involvement of factors besides VEGF in neovascularization. The composition may additionally contain other agents including, but not limited to, transforming growth factor β (TGFβ), interleukin-10 (IL-10), aspirin, a vitamin, and/or an antineoplastic agent.

An effective amount of anti-VEGF agent, either as the sole active agent, or with one or more other non-antiinflammatory agents as previously described, is administered. Administration of either agent may be by any route, and the agents may be administered by the same route or by different routes, including enteral, parental, and ocular routes such as intravitreal injection, subconjunctival injection, retrobulbar injection, topical, etc. As one example, the anti-VEGF agent (bevacizumab, sunitinib, etc.) may be topically administered to intact or compromised eyes, skin, mucous membranes, etc. to reduce scarring after trauma, surgery, radiation, burns, wounds, etc. As another example, it may be locally administered to a site in a surgical field to ameliorate inflammation (e.g., adhesions, scarring, effusions) of pleura, epicardium, etc. after thoracic, cardiac, abdominal, etc. surgery. As another example, it may be administered intrathecally (brain, spinal cord, etc.). As another example, it may be administered by inhalation, for example, to ameliorate inflammation in the respiratory tract (nose, trachea, bronchi, lungs, etc.). As another example, it may be instilled in a body cavity (ventricles, sinuses, bladder, etc.). As another example, sunitinib may be administered systemically (e.g., a single dose/week for one month, then monthly reevaluation of need) or topically (e.g., from about 10 ng/ml to about 100 ng/ml), or intraocularly (e.g., from about 7 ng/ml to about 20 μg/ml). In one embodiment, the administered dose of bevacizumab is less than about 5 mg/0.1 ml. In another embodiment, the administered dose of bevacizumab ranges from 0.1 mg/ml to about 50 mg/mi. In another embodiment, the dose of bevacizumab administered systemically ranges from about 0.05 mg/ml to about 5 mg/ml. In one embodiment, the dose of bevacizumab administered intraocularly (e.g., intravitreally) is about 0.005 mg/0.1 ml to about 5 mg/0.1 ml. In one embodiment, the dose of bevacizumab administered topically to the eye is up to 5 mg/ml, and in another embodiment it may be higher. While these doses recite bevacizumab, one skilled in the art will appreciate that they may be used with other anti-VEGF agents, and that doses for a specific agent may be determined empirically, by patient disease severity, other patient variables, etc.

Solutions may be prepared using a physiological saline solution as a vehicle. The pH of an ophthalmic solution may be maintained at a substantially neutral pH (for example, about 7.4, in the range of about 6.5 to about 7.4, etc.) with an appropriate buffer system as known to one skilled in the art (for example, acetate buffers, citrate buffers, phosphate buffers, borate buffers).

The formulations may also contain pharmaceutically acceptable excipients known to one skilled in the art such as preservatives, stabilizers, surfactants, chelating agents, antioxidants such a vitamin C, etc. Preservatives include, but are not limited to, benzalkonium chloride, chlorobutanol, thimerosal, phenylmercuric acetate and phenylmercuric nitrate. A surfactant may be Tween 80. Other vehicles that may be used include, but are not limited to, polyvinyl alcohol, povidone, hydroxypropyl methyl cellulose, poloxamers, carboxymethyl cellulose, hydroxyethyl cellulose, purified water, etc. Tonicity adjustors may be included, for example, sodium chloride, potassium chloride, mannitol, glycerin, etc. Antioxidants include, but are not limited to, sodium metabisulfite, sodium thiosulfate, acetylcysteine, butylated hydroxyanisole, butylated hydroxytoluene, etc. In one embodiment, bevacizumab and/or other anti-VEGF agent(s) may be administered via a controlled release system (i.e., delayed release formulations and/or extended release formulations) such as polylactic or polyglycolic acid, silicone, hema, and/or polycaprolactone microspheres, microcapsules, microparticles, nanospheres, nanocapsules, nanoparticles, etc. A slow release system may release about 10 ng anti-VEGF agentday to about 50 ng anti-VEGF agent/day for an extended period.

In various embodiments, the compositions may contain other agents. The indications, effective doses, formulations, contraindications, vendors, etc. of these are available or are known to one skilled in the art. It will be appreciated that the agents include pharmaceutically acceptable salts and derivatives.

Administration of an anti-VEGF agent such as bevacizumab, and optionally other agents such as an anti-PDGF agent, another anti-VEGF agent, etc., may supplement or replace PDT and hence avoid the retinal damage frequently associated with PDT. PDT is frequently used to reduce or prevent damage from leaky vessels associated with age related macular degeneration and other diseases. A series of PDT treatments is often performed with a cumulative effect that, over time, results in retinal damage which in some cases may be severe. The present invention may obviate the need for PDT thus eliminating its associated damage.

Bevacizumab at a dose of 5 mg/0.1 ml has been found not to be toxic. In embodiments where bevacizumab or another anti-VEGF agent is administered as the sole agent to ameliorate inflammation, the dose of bevacizumab ranges between about 0.01 mg/0.1 ml to about 5 mg/0.1 ml.

Bevacizumab may be used to ameliorate (e.g., reduce, prevent, slow, etc.) corneal neovascularization. The following example demonstrates the efficacy of bevacizumab on comeal neovascularization that was chemically induced. One skilled in the art, however, appreciates that the invention is not so limited and is applicable to amelioration of corneal neovascularization resulting from other etiologies. These include, but are not limited to, corneal transplant rejection, mechanical trauma, corneal ulcers caused by any mechanism including microorganisms, conjunctivitis sicca, use of contact lenses, presence of a foreign body, pemphigus, Sjorgen's disease, and other auto immune diseases of the cornea and/or sclera.

### EXAMPLE 1

Sixteen Male Long Evans pigmented rats (200 g to 250 g) were administered general anesthesia (94.7 mg/kg ketamine hydrochloride/xylazine i.p.) supplemented by topical anesthesia (0.5% proparacaine hydrochloride). One cornea of each animal was cauterized by pressing an applicator stick (1.8 mm diameter) coated with 75% silver nitrate/25% potassium nitrate (Arzol Chemical Co., Keen, N.H.) to the central cornea for ten seconds under the operating microscope. Excess silver nitrate was removed by rinsing the eyes with balanced salt solution (5 ml) and gentle blotting with tissue paper. To increase the reproducibility of the injuries, a single investigator cauterized all animals.

Animals were randomized to one of two groups: group 1 (n=10) received topical 4 mg/ml bevacizumab, and group 2 (n=6) received saline. Both treatments were topically administered two times per day for seven days, and began immediately after cauterization. Corneas from anesthetized animals were evaluated by slit-lamp biomicroscopy on the third and sixth day. Corneal photographs were taken with ×25 magnification using a camera attached to the slit-lamp microscope (Topcon SL-7E, Tokyo Japan) on the seventh day. Neovascularization in each cornea was evaluated by an examiner blinded as to the treatment groups. For each eye, the extent of bum stimulus response was scored as follows: 0 (no blister, not raised above comeal surface), +1 (small blister, raised slightly above the surface), +2 (medium blister, raised moderately above the surface), +3 (large blister). Only corneas with a burn stimulus score of +2 or higher were included for the calculation of the mean burn stimulus and neovascularization scores in each group. All photographs were converted to high-resolution digital forms by scanner (Cano scan 9900F, Canon, Tokyo Japan). The corneal surface covered with neovascular vessels was measured on the photographs as the percentage of the total area of the cornea. Image analysis was performed on each cornea using an image processing and analysis software program (Image J 1.31v. -Wayne Rasband at the Research Services Branch, National Institute of Mental Health, Bethesda Md.). The area of neovascularization was measured in terms of pixels and its ratio to the entire corneal area was determined as the percentage of corneal neovascularization. A drawing of comeal blood vessels was made by one investigator to compare with digital photos and to ensure that no vascular area was missed during calculation of percent area. After scoring the burn stimulus and the percentage of neovascularization for both groups, the animals were sacrificed on the seventh day.

Following sedation (previously described), enucleation was performed before the animals were euthanized. Immediately after enucleation, the globes were penetrated with a 27-gauge needle, 1.0 mm from the limbus at the 3 and 9 o'clock meridians which allowed the fixative to rapidly fill the eyes. The eyes were prepared for histologic examination using 10% formaldehyde. After fixation for twenty-four hours, the eyes were removed from the fixative. Corneas were dehydrated, sectioned, soaked in xylene and paraffin, embedded in paraffin, and cut at 1 μm for staining with hematoxylin and eosin (H&E) for light microscopy.

Light microscopic examination was performed on every microscopic section. Sections were examined by dividing the corneas into two halves through the center of the lesion and were evaluated with regard to the intensity of new vessels, polymorphonuclear (PMN) leucocytes, edema, and fibroblastic activity.

The Mann-Whitney U test was used for comparisons. Statistical significance was defined as a probability (p) of less than 0.05 of the result being due to chance alone.

The burn stimulus score was +2 or higher in all eyes. The mean burn stimulus scores were not statistically different between the treatment and the placebo groups (P>0.05, Mann-Whitney U test).

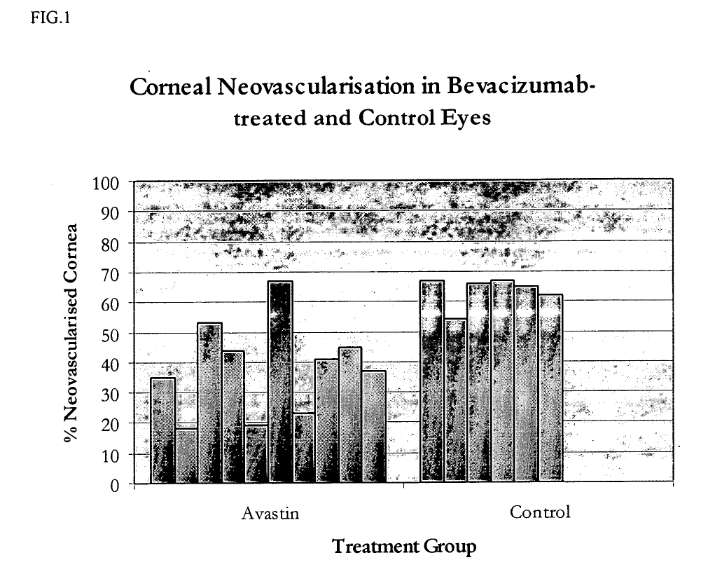

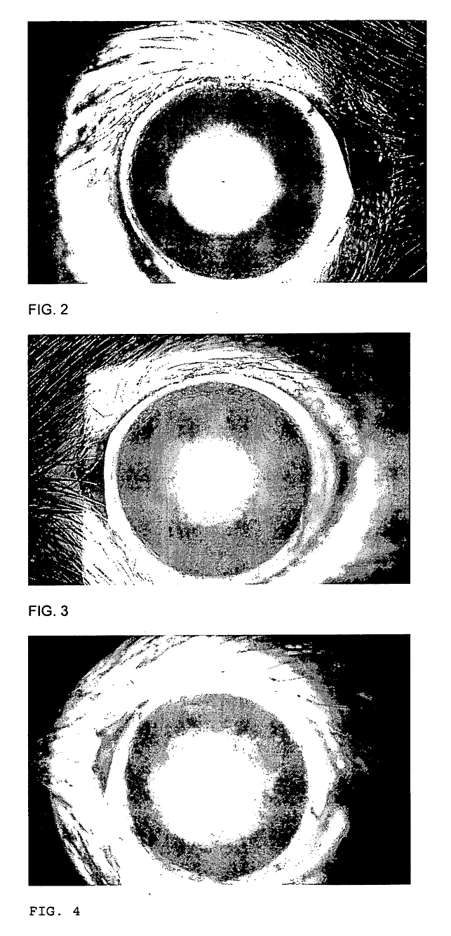

FIG. 1 shows normalized areas of corneal neovascularization in bevacizumab-treated eye (n=10) and control eyes (n=6). FIGS. 2 and 3 are photographs of representative bevacizumab-treated eyes, and FIG. 4 is a photograph of a representative control eye. The difference was statistically significant (p<0.02, Mann Whitney test). As seen in FIG. 1, bevacizumab-treated eyes had less corneal neovascularization than control eyes. In bevacizumab-treated eyes, corneal neovascularization covered, on average, 38.2 ± 15.5% (mean ± standard deviation (SD) of the corneal surface. In control eyes, corneal neovascularization covered, on average, 63.5 ± 5.0% (mean ± SD) of the corneal surface (p<0.02, Mann-Whitney test). Topically administered bevacizumab at 4 mg/ml decreased corneal neovascularization by 40%.

### EXAMPLE 2

Animals are treated and prepared as in Example 1, expect that bevacizumab is administered by intravitreal injection using a 30 g needle. Corneal neovascularization in treated eyes is reduced over untreated eyes.

### EXAMPLE 3

Animals are treated and prepared as in Example 1, expect that bevacizumab is administered by subconjunctival injection using a 30 g needle. Corneal neovascularization in treated eyes is reduced over untreated eyes.

### EXAMPLE 4

Animals are treated and prepared as in Example 1, expect that bevacizumab is administered by subretinal injection using a 30 g needle. Comeal neovascularization in treated eyes is reduced over untreated eyes.

### EXAMPLE 5

Animals are treated and prepared as in Example 1, expect that bevacizumab is administered by retrobulbar injection using a 30 g needle. Comeal neovascularization in treated eyes is reduced over untreated eyes.

### EXAMPLE 6

Animals are treated and prepared as in Example 1, expect that bevacizumab is replaced by ranibizumab. Comeal neovascularization in treated eyes is reduced over untreated eyes.

### EXAMPLE 7

Animals are treated and prepared as in Example 2, expect that bevacizumab is replaced by ranibizumab. Comeal neovascularization in treated eyes is reduced over untreated eyes.

### EXAMPLE 8

Animals are treated and prepared as in Example 3, expect that bevacizumab is replaced by ranibizumab. Comeal neovascularization in treated eyes is reduced over untreated eyes.

### EXAMPLE 9

Animals are treated and prepared as in Example 4, expect that bevacizumab is replaced by ranibizumab. Comeal neovascularization in treated eyes is reduced over untreated eyes.

### EXAMPLE 10

Animals are treated and prepared as in Example 5, expect that bevacizumab is replaced by ranibizumab. Corneal neovascularization in treated eyes is reduced over untreated eyes.

### EXAMPLE 11

Animals are treated and prepared as in Example 1, expect that bevacizumab is replaced by pegaptanib. Corneal neovascularization in treated eyes is reduced over untreated eyes.

### EXAMPLE 12

Animals are treated and prepared as in Example 2, expect that bevacizumab is replaced by pegaptanib. Corneal neovascularization in treated eyes is reduced over untreated eyes.

### EXAMPLE 13

Animals are treated and prepared as in Example 3, expect that bevacizumab is replaced by pegaptanib. Corneal neovascularization in treated eyes is reduced over untreated eyes.

### EXAMPLE 14

Animals are treated and prepared as in Example 4, expect that bevacizumab is replaced by pegaptanib. Corneal neovascularization in treated eyes is reduced over untreated eyes.

### EXAMPLE 15

Animals are treated and prepared as in Example 5, expect that bevacizumab is replaced by pegaptanib. Corneal neovascularization in treated eyes is reduced over untreated eyes.

### EXAMPLE 16

Animals are treated and prepared as in Example 1, expect that bevacizumab is replaced by sunitinib maleate. Corneal neovascularization in treated eyes is reduced over untreated eyes.

### EXAMPLE 17

Animals are treated and prepared as in Example 2, expect that bevacizumab is replaced by sunitinib maleate. Corneal neovascularization in treated eyes is reduced over untreated eyes.

### EXAMPLE 18

Animals are treated and prepared as in Example 3, expect that bevacizumab is replaced by sunitinib maleate. Corneal neovascularization in treated eyes is reduced over untreated eyes.

### EXAMPLE 19

Animals are treated and prepared as in Example 4, expect that bevacizumab is replaced by sunitinib maleate. Corneal neovascularization in treated eyes is reduced over untreated eyes.

### EXAMPLE 20

Animals are treated and prepared as in Example 5, expect that bevacizumab is replaced by sunitinib maleate. Corneal neovascularization in treated eyes is reduced over untreated eyes.

### EXAMPLE 21

Animals are treated and prepared as in any of Example 1-20, expect that the agent is administered using an intraocular device, such as the device described in the co-pending related application that has been expressly incorporated by reference herein. Comeal neovascularization in treated eyes is reduced over untreated eyes.

It should be understood that the embodiments of the present invention shown and described in the specification are only preferred embodiments of the inventor who is skilled in the art and are not limiting in any way. As one example, the inventive method may be used to treat cerebral edema associated with meningitis by intravenously administering bevacizumab. Therefore, various changes, modifications or alterations to these embodiments may be made or resorted to without departing from the spirit of the invention and the scope of the following claims.

What is claimed is:

1. A method of ameliorating inflammation in a patient, the method comprising providing to the patient in need thereof a biocompabble composition comprising an anti-vascular endothelial growth factor (VEGF) agent selected from at least one of bevacizumab, ranibizumab, pegaptanib, anti-VEGF siRNA, TNP470, integrin av antagonists, 2-methoxyestradiol, paclitaxel, P38 mitogen activated protein kinase inhibitors, or sunitinib maleate in the absence of an anti-inflammatory agent.

2. A therapeutic method comprising providing to at least one inflammatory tissue in a patient a biocompatible composition containing an anti-VEGF agent selected from at least one of bevacizumab, ranibizumab, pegaptanib, TNP470, integrin av antagonists, 2-methoxyestradiol, paclitaxel, P38 mitogen activated protein kinase inhibitors, or sunitinib maleate in the absence of an anti-inflammatory agent, the method ameliorating inflammation in the absence of angiogenesis.

3. The method of claim 2 wherein inflammation in the absence of angiogenesis is from at least one of surgery, inflammatory diseases of the central nervous system, conditions resulting in cerebral edema, macular edema, or inflammatory diseases of the eye.

4. The method of either claim 1 or claim 2 wherein inflammation is a result of at least one of an immune disease, a microbial infection, trauma, ischemic diseases, diabetes, age related macular degeneration, retinitis pigmentosa, allergy, or a degenerative diseases.

5. The method of either claim 1 or claim 2 wherein the patient has at least one of synovitis, uveitis, iritis, retinal vasculitis, optic nerve neuritis, papillitis, or diabetic retinopathy.

6. The method of either claim 1 or claim 2 wherein the anti-VEGF agent ameliorates at least one of scars or adhesions.

7. The method of either claim 1 or claim 2 wherein the anti-VEGF agent is administered is by a route selected from at least one of enteral, parental, ocular, topical, intrathecal, inhalation, or instillation.

8. The method of either claim 1 or claim 2 wherein the body site is at least one of an eye, lung, bone, brain, joint, heart, or muscle.

9. The method of either claim 1 or claim 2 wherein the dose of anti-VEGF agent is less than about 5 mg/0.1 ml.

10. The method of either claim 1 or claim 2 wherein the dose of anti-VEGF agent ranges from 0.1 mg/ml to about 50 mg/ml.

11. The method of either claim 1 or claim 2 wherein the anti-VEGF agent is administered systemically at a dose from about 0.05 mg/ml to about 5 mg/ml.

12. The method of either claim 1 or claim 2 wherein the anti-VEGF agent is administered intraocularly at a dose from about 0.005 mg/0.1 ml to about 5 mg/0.1 ml.

13. The method of either claim 1 or claim 2 wherein the anti-VEGF agent is administered topically to the eye at a dose up to about 5 mg/ml.

14. The method of either claim 1 or claim 2 wherein the dose of the anti-VEGF agent ranges from about 0.01 mg/0.1 ml to about 5 mg/0.1 ml.

15. The method of either claim 1 or claim 2 wherein the anti-VEGF agent is formulated in at least one of microspheres, nanospheres, microcapsules, or nanocapsules.

16. The method of either claim 1 or claim 2 wherein the anti-VEGF agent is a controlled release formulation.

17. A method to ameliorate corneal neovascularization comprising ocularly administering an anti-vascular endothelial growth factor agent at a concentration ranging between 0.01 mg/0.1 ml to about 5 mg/0.1 ml for a duration sufficient to ameliorate neovascularization.

18. The method of claim 17 wherein the agent is at least one of bevacizumab, ranibizumab, pegaptanib, sunitinib maleate, anti-VEGF siRNA, TNP470, integrin av antagonists, 2-methoxyestradiol, paclitaxel, or P38 mitogen activated protein kinase inhibitors.

19. The method of claim 17 wherein ocular administration is selected from topical, intraocular injection, or intraocular implantation.

20. A method to ameliorate corneal neovascularization comprising topically administering to an eye of a patient in need thereof a biocompatible composition comprising bevacizumab at a concentration up to about 5 mg/0.1 ml for a duration sufficient to ameliorate neovascularization.
